(19) United States
(12) Patent Application Publication
(10) Pub. No.: US 2026/0159553 A1
(43) Pub. Date: Jun. 11, 2026

(54) MULTICISTRON EXPRESSION VECTOR FOR COVID-19 VACCINE

(71) Applicant: Kashiv BioSciences, LLC, Piscataway, NJ (US)

(72) Inventors: Sudharti Gupta, Ahmedabad (IN); Om Narayan, Ahmedabad (IN); Narahari Ponnapu Reddy, Faridabad (IN); Chandramauli Rawal, Piscataway, NJ (US)

(21) Appl. No.: 18/707,293

(22) PCT Filed: Nov. 3, 2022
(86) PCT No.: PCT/IB2022/060586

§ 371 Date: May 3, 2024

(30) Foreign Application Priority Data
Nov. 3, 2021 (IN) 202121050472

(51) Int. Cl. C07K 14/005; A61K 39/00; A61K 39/215; A61P 37/04; B01D 15/38; C12N 15/85

(52) U.S. Cl. CPC C07K 14/005; A61K 39/215; A61P 37/04; B01D 15/3819; C12N 15/85; A61K 2039/53; C12N 2770/20022; C12N 2770/20034; C12N 2770/20051; C12N 2830/50; C12N 2840/206

(57) ABSTRACT

The present invention provides an expression vector comprises gene of interest encode more than one structural protein to enhance immune responses against Severe Acute Respiratory Syndrome Coronavirus 2 (SARS-COV-2) and its variants. Furthermore, the expression vector to produce mRNA expresses more than one structural protein to generate immune response against Severe Acute Respiratory Syndrome Coronavirus 2 (SARS-COV-2) and its variants.

### FIELD OF INVENTION

The present invention provides an expression vector comprises gene of interest encode more than one structural protein to enhance immune responses against severe acute respiratory syndrome coronavirus 2 (SARS-COV-2) and its variants.

Furthermore, the vector construct comprises regulatory elements selected from promoter, Internal ribosome entry site (IRES), untranslated regions, gene of interest which includes proteins of severe acute respiratory syndrome coronavirus 2 (SARS-COV-2) selected from spike(S), envelope (E), membrane (M), and nucleocapsid (N).

### BACKGROUND OF INVENTION

Vaccines are biological preparations required to initiate an immune response against antigens specifically provides prophylactic or therapeutic efficacy to disease.

Coronaviruses are a group of related RNA viruses causes mild to lethal respiratory tract infections in mouse, pig, cat, dog, birds and mammals. Coronaviruses from the subfamily Orthocoronavirinae, in the family Coronaviridae, order Nidovirales. Coronavirus is a single-stranded, positive-stranded RNA with a complete genome with its length between 26 and 32 kb. In the past years, human coronavirus vividly affected human population with a total of 7 coronaviruses i.e., Human coronavirus 229E (HCoV-229E) and HCoV-OC43 discovered in the 1960s, SARS-COV that appeared in 2003, and HCoV-NL63 that was isolated in the Netherlands in 2004, HCoV-HKU1 identified in Hong Kong in 2005, new middle east respiratory syndrome (Middle East respiratory syndrome virus, MERS) coronavirus MERS-COV that appeared in the Middle East in 2012 and the global outbreak of SARS-COV-2 (2019-nCOV) that appeared in 2019. The first genome sequence of a SARS-COV-2 isolate (Wuhan-Hu-1) was released by investigators from the Chinese CDC in Beijing on Jan. 10, 2020 at Virological, a UK-based discussion form for analysis and interpretation of virus molecular evolution and epidemiology. The sequence was then deposited in GenBank on Jan. 12, 2020, having Genbank Accession number MN908947.1. The emerging variants of SARS-COV-2 alpha, beta, gamma, delta were declared as variant of concern. The spike protein (S protein) of the SARS-COV recognizes the receptor protein ACE2 (angiotensin-converting enzyme 2) on the cell membrane finally mediates and promotes the fusion of the viral envelope and the cell membrane leads to highly pathogenic infection. The severe acute respiratory syndrome coronavirus (SARS-COV-2 and SARS) have a high degree of homology. The SARS-COV-2 have sixteen non-structural proteins (NSPs) involved in genome replication and early transcription regulation. The SARS-COV-2 has four structural proteins which includes spike(S), envelope (E), membrane (M), and nucleocapsid (N). The surface protein, or spike protein of SARS-COV-2 is the foremost with two subunits S1 and S2, the S1 includes mainly a Receptor Binding Domain (RBD) that recognizes cellular receptors. The structural proteins also hold the potential to develop as vaccine candidate for generating neutralizing antibodies in subject.

The inactivated or traditional vaccine has fifty to seventy percent of effectiveness against variants of the virus. The advanced SARS-COV-2 mRNA vaccine encodes a single protein of virus that isn't effective against mutated strain therefore it may fail and its coverage for upcoming variants is also limited. The vaccine elicits an immune response in a specific manner toward its encoded protein. An emerging scope is warranted to advance messenger ribonucleic acid (mRNA) vaccine that can encode more than one protein in a single construct and maximize its potential to neutralize the virus and upcoming mutated variants by induing strong immune responses.

The present invention determines an expression vector design encoding more than one structural protein of severe acute respiratory syndrome coronavirus 2 (SARS-COV-2) for the preparation of mRNA vaccine as prophylactic or therapeutic medium against viral infection caused by SARS-CoV-2.

### SUMMARY OF INVENTION

The present invention discloses the novel expression vector construct to produce mRNA vaccine comprises a gene of interest encoding more than one structural protein of severe acute respiratory syndrome coronavirus 2 (SARS-COV-2) and its variants.

The present invention discloses the novel expression vector construct to produce mRNA vaccine comprises a gene of interest encoding the structural proteins of severe acute respiratory syndrome coronavirus 2 (SARS-COV-2) and its variants. The construct includes nucleic acid molecule as a part of vaccine composition that has immunogenicity to trigger immune response.

In an embodiment, the present invention discloses the novel expression vector construct to produce mRNA vaccine encoding spike(S), envelope (E), membrane (M), and nucleocapsid (N) proteins of SARS-COV-2.

In an embodiment, the present invention comprises vector construct with regulatory and non-regulatory elements. In another embodiment, the present invention discloses an engineered expression vector comprising an origin of replication, a multicloning site, and a selectable marker.

In an embodiment, the expression vector comprises elements selected from core promoters, proximal promoters, distal enhancers, silencers, insulators/boundary elements, and locus control regions.

In an embodiment, the invention related to the preparation of expression vector construct comprises;
a) promoter sequence;
b) untranslated regions (3′ UTR and 5′ UTR);
c) internal Ribosome Entry Site (IRES) element;
d) gene of interest encoding more than one structural protein of SARS-COV-2; and
e) polyadenylation (Poly A) tail.

In an embodiment, the expression vector encoding more than one structural proteins of severe acute respiratory syndrome coronavirus 2 (SARS-COV-2) comprising;
a) T7 promoter;
b) 5′ Untranslated region (5′ UTR);
c) spike protein of SARS-COV-2 delta variant;
d) internal Ribosome Entry Site (IRES) element;
e) envelope protein of SARS-COV-2 delta variant;
f) internal Ribosome Entry Site (IRES) element;
g) membrane protein of SARS-COV-2 delta variant;
h) internal Ribosome Entry Site (IRES) element;
i) nucleocapsid protein of SARS-COV-2 delta variant;
j) 3′ Untranslated region (3′ UTR); and
k) polyadenylation (Poly A) tail.

In an embodiment, an expression vector construct encoding more than one structural protein of severe acute respiratory syndrome coronavirus 2 (SARS-COV-2) comprising:
a) promoter sequence;
b) untranslated regions;
c) more than one structural protein selected from spike(S), envelope (E), membrane (M), and nucleocapsid (N);
d) one or more internal Ribosome Entry Site (IRES) element; and
e) polyadenylation (poly A) tail.

In an embodiment, an expression vector construct encoding more than one structural protein of severe acute respiratory syndrome coronavirus 2 (SARS-COV-2) comprising:
a) promoter sequence;
b) 5′ untranslated region (5′ UTR);
c) internal ribosomal entry sites (IRES) element;
d) one or more structural proteins of SARS-COV-2 variant selected from spike(S), envelope (E), membrane (M), and nucleocapsid (N);
e) internal ribosomal entry sites (IRES) element;
f) 3′ untranslated region (3′ UTR); and
g) polyadenylation (Poly A) tail.

In an embodiment, the invention related to the preparation of expression vector construct comprises;
a) promoter sequence;
b) untranslated regions (3′ UTR and 5′ UTR);
c) gene of interest encoding more than one structural protein selected from spike(S), envelope (E), membrane (M), and nucleocapsid (N);
d) internal Ribosome Entry Site (IRES) element; and
e) polyadenylation (Poly A) tail.

### BRIEF DESCRIPTION OF FIGURES

FIG. 1: depicts schematic representation of mRNA vaccine vector construct that includes T7 promoter, 5′ untranslated region (5′ UTR), spike protein of SARS-COV-2 delta variant, envelope protein of SARS-COV-2 delta variant, membrane protein of SARS-COV-2 delta variant, nucleocapsid protein of SARS-COV-2 delta variant, Internal Ribosome Entry Site (IRES) element, 3′ untranslated region (3′ UTR) and Polyadenylation tail (PolyA).

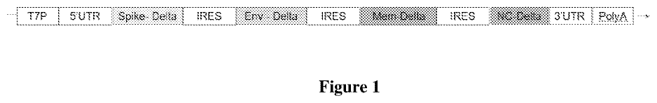

FIG. 2: depicts schematic representation of multicistron vector map encoding spike protein of SARS-COV-2 delta variant, envelope protein of SARS-COV-2 delta variant, membrane protein of SARS-COV-2 delta variant, nucleocapsid protein of SARS-COV-2 delta variant and other elements enclosed in construct.

FIG. 3: depicts schematic representation of RNA Marker and IVT mRNA (8 kb) in agarose gel electrophoresis.

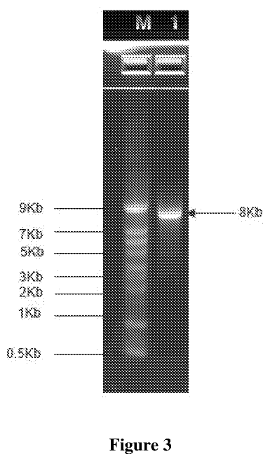

FIG. 4: depicts in-vitro cell-based expression of mRNA.

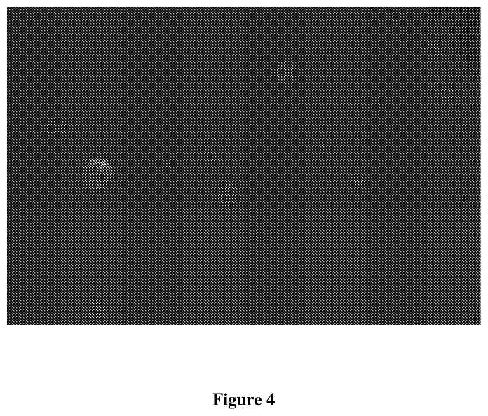

### DETAILED DESCRIPTION OF INVENTION

The present invention discloses the novel expression vector construct to produce mRNA vaccine comprises gene of interest encoding more than one structural protein of severe acute respiratory syndrome coronavirus 2 (SARS-COV-2) and its variants. The construct includes nucleic acid molecule as a part of vaccine composition that has immunogenicity to trigger immune response.

### Expression Vector Construct

The term “expression vector construct” or “expression construct” or “expression vector” or “Plasmid” or “Plasmid DNA” or “Plasmid DNA vector” are interchangeable and refers to the nucleotide sequences of the invention containing the nucleotide sequences to be expressed. The restriction sites between the 5′ and 3′ ends of the sequences allows insertion, removal of sequences in a vector. The expression vector construct includes nucleotide sequence, transcriptional and translational control sequences, such as a promoter and/or termination sequences, operably linked to the nucleotide sequence and allowing expression in a host cell.

“Plasmid” or “Plasmid DNA vector” or “Plasmid DNA” used in mRNA vaccine production as are easy to replicate (copy) and reliable containing the target gene sequence. Plasmid DNA vector comprises a multiple cloning site, an RNA promoter sequence, optionally a selection marker, such as an antibiotic resistance factor, and a sequence suitable for multiplication of the vector, such as an origin of replication. The plasmid backbone is pHSG298 in the context of the present invention.

### Promoter

The term “promoter” in the present invention refers to a sequence of nucleic acid required to turn a gene on or off. The RNA polymerase After the recognition of the promoter region by an RNA polymerase, the binding of the RNA polymerase becomes an initiation complex.

The T7 promoter sequence is a sequence of 18 base pairs long up nucleotides to transcription start site recognized by T7 RNA polymerase used to regulate gene expression of recombinant proteins.

### IRES

The term “IRES” or “internal ribosome entry site” refers to an RNA element that allows for translation initiation in a cap-dependent manner and cap-independent manner, as part of the greater process of protein synthesis. A vector construct can contain a multiple IRES element.

### 5′ UTR

The term “5′ untranslated region” or “5′ UTR” refers as a specific part of messenger RNA (mRNA). The 5 ‘UTR is located 5’ of the open reading frame of the mRNA. The 5′ UTR can contain elements for controlling gene expression. The 5′UTR begins at the transcription start site and ends one nucleotide before the start codon of the open reading frame. The 5 ‘UTR can be modified by adding a cap after transcription.

### 3’ UTR

The term “3 ‘untranslated region” or “3’ UTR” refers to a mRNA that is located between the region encoding the protein and the poly(A) sequence. The 3′UTR sequences transcribed into their respective mRNAs encoded by genes during the gene expression process and not further translated into an amino acid sequence. The 3′UTR sequence can be an RNA sequence, or a DNA sequence.

### Vaccine

The term “vaccine” or “immunological preparation” refers to a biological composition comprising an immunogen or antigenic molecule that stimulates immune response in body to generate prophylactic or therapeutic effect against disease causing antigen.

mRNA

The term “mRNA” or “messenger RNA” refers to nucleic acid sequence with at least one open reading frame that can be translated by a cell or organism comprising the mRNA.

### GOI

The term “GOI” or “gene of interest” refers to nucleic acid sequence encoding structural proteins which are selected from spike protein(S), envelope protein (E), membrane protein (M), nucleocapsid protein (N or NC) of SARS-COV-2; for example, the N protein holds the RNA genome, and the S, E, and M proteins together create the viral envelope. The GOI can be obtained from a variety of sources, including source of interest or synthesizing from known or predicted sequence information, and may include sequences designed to have desired parameters.

### Chromatography

The term “Chromatography” used herein for the separation of biomolecule such as plasmid DNA and mRNA from mixture based on size, surface charge and/or interaction the purification method is selected from the group consisting of cation exchange chromatography, anion exchange chromatography, membrane absorbers, reversed phase chromatography, normal phase chromatography, size exclusion chromatography, hydrophobic interaction chromatography, mixed mode chromatography, affinity chromatography, hydroxylapatite (HA) chromatography, HPLC, core bead chromatography or combinations thereof. The Ion exchange chromatography or IEX refers to the separation of molecules on the basis of differences in their net surface charge. The Affinity chromatography, the separation based on interactions between two molecules, purification by placing one of the interacting molecules, referred to as affinity ligand, onto a solid matrix to create a stationary phase while the target molecule is in the mobile phase. The Mixed/Multi-mode chromatography or “MMC” utilize more than one form of interaction between the stationary phase and analytes in order to achieve the separation, with multiple retention modes on a single MMC column provides additional dimension to a separation method by its unique selectivity and retention of a variety of compounds, especially polar and charged molecules.

In an embodiment, the gene of interest encodes at least two structural proteins.

In an embodiment, the gene of interest encodes at least three structural proteins.

In an embodiment, the invention related to the preparation of expression vector construct comprises;
a) promoter sequence;
b) untranslated regions (3′ UTR and 5′ UTR);
c) internal Ribosome Entry Site (IRES) element;
d) gene of interest encoding more than one structural protein of SARS-COV-2; and
e) polyadenylation (Poly A) tail.

In an embodiment, wherein the gene of interest comprises structural proteins of severe acute respiratory syndrome coronavirus 2 (SARS-COV-2) and its variants.

In an embodiment, wherein the gene of interest comprises spike(S) protein of severe acute respiratory syndrome coronavirus 2 (SARS-COV-2) and its variants.

In an embodiment, wherein the gene of interest comprises nucleocapsid (N) protein of severe acute respiratory syndrome coronavirus 2 (SARS-COV-2) and its variants.

In an embodiment, wherein the gene of interest comprises envelope (E) protein of severe acute respiratory syndrome coronavirus 2 (SARS-COV-2) and its variants.

In an embodiment, wherein the gene of interest comprises membrane (M) protein of severe acute respiratory syndrome coronavirus 2 (SARS-COV-2) and its variants.

In one embodiment, the expression vector encoding more than one structural proteins of severe acute respiratory syndrome coronavirus 2 (SARS-COV-2) comprising:
a) T7 promoter;
b) 5′ Untranslated region (5′ UTR);
c) spike protein of SARS-COV-2 delta variant;
d) internal Ribosome Entry Site (IRES) element;
e) envelope protein of SARS-COV-2 delta variant;
f) internal Ribosome Entry Site (IRES) element;
g) membrane protein of SARS-COV-2 delta variant;
h) internal Ribosome Entry Site (IRES) element;
i) nucleocapsid protein of SARS-COV-2 delta variant;
j) 3′ Untranslated region (3′ UTR); and
k) polyadenylation (Poly A) tail.

In an embodiment, the invention related to the preparation of expression vector construct comprises;
a) promoter sequence;
b) untranslated regions;
c) gene of interest more than one structural protein selected from spike(S), envelope (E), membrane (M), and nucleocapsid (N);
d) internal Ribosome Entry Site (IRES) element; and
e) polyadenylation (Poly A) tail.

In an embodiment, the invention related to a process for the preparation of vaccine vector construct encoding more than one antigenic polypeptide comprises;
a) promoter sequence to initiate the reaction for expression of polypeptide;
b) 5′ UTR Untranslated region to serve as entry point for the ribosome during translation;
c) internal Ribosome Entry Site (IRES) element initiates translation in a cap-dependent and independent manner;
d) 3′ UTR Untranslated region regulates mRNA-based processes;
e) poly(A) tail provides stability and allows mature messenger RNA molecule to export.

In an embodiment, the invention related to the preparation of expression vector construct comprises;
a) promoter sequence
b) 5′ UTR Untranslated region;
c) encoding one antigenic polypeptide of SARS-COV-2;
d) internal Ribosome Entry Site (IRES) element;
e) encoding another antigenic polypeptide of SARS-COV-2;
f) 3′ UTR Untranslated region; and
g) polyadenylation (Poly A) tail.

In an embodiment, the invention provides a process for the preparation of mRNA from vector construct comprises;
a) selection and identification of gene of interest for various antigen;
b) cloning the selected gene of interest in a vector;
c) isolation of suitable plasmid;
d) linearization of isolated plasmid;
e) performing the in-vitro reaction for the preparation mRNA copies;
f) purification of mRNA by chromatography methods.

In an embodiment, a process for the purification of mRNA comprises;
a) transfect the expression vector as claimed into E. Coli host cells;
b) harvesting of cells followed by lysis, neutralization, and precipitation;
c) isolation and purification of plasmid;
d) linearization of purified plasmid;
e) performing the in-vitro transcription reaction for the preparation mRNA;
f) purification of mRNA by chromatography methods.

In an embodiment, an expression vector construct encoding more than one structural protein of severe acute respiratory syndrome coronavirus 2 (SARS-COV-2) comprising:
a) promoter sequence;
b) untranslated regions;
c) more than one structural protein selected from spike(S), envelope (E), membrane (M), and nucleocapsid (N);
d) one or more internal Ribosome Entry Site (IRES) element; and
e) polyadenylation (poly A) tail.

In an embodiment, an expression vector construct encoding more than one structural protein of severe acute respiratory syndrome coronavirus 2 (SARS-COV-2) comprising:
a) promoter sequence;
b) 5′ untranslated region (5′ UTR);
c) internal ribosomal entry sites (IRES) element;
d) one or more structural proteins of SARS-COV-2 variant selected from spike(S), envelope (E), membrane (M), and nucleocapsid (N);
e) internal ribosomal entry sites (IRES) element;
f) 3′ untranslated region (3′ UTR); and
g) polyadenylation (Poly A) tail.

In an embodiment, the expression vector construct expresses the mRNA capable to encode one or more structural protein provides immune response against SARS-COV-2 virus.

In an embodiment, a process for the purification of mRNA comprises;
a) transfect the expression vector into E. Coli host cells;
b) harvesting of cells followed by lysis, neutralization and precipitation;
c) isolation and purification of plasmid;
d) linearization of purified plasmid;
e) performing the in-vitro reaction for the preparation mRNA;
f) purification of mRNA by chromatography methods.

In an embodiment, the expression construct comprises as gene of interest is SARS-COV-2 protein.

In another embodiment, the expression construct comprises as gene of interest selected from spike protein, nucleocapsid protein, membrane protein.

In an embodiment, the expression construct comprises as gene of interest is spike protein.

In an embodiment, the expression construct comprises as gene of interest is Receptor binding domain of spike(S) protein.

In another embodiment, the spike(S) protein of SARS-COV-2 variant selected from alpha, beta, gamma, delta, eta, Iota, kappa, lambda, and Mu.

In an embodiment, the expression construct comprises as gene of interest is nucleocapsid protein.

In another embodiment, the nucleocapsid (N) protein of SARS-COV-2 variant selected from alpha, beta, gamma, delta, eta, Iota, kappa, lambda and Mu.

In an embodiment, the expression construct comprises as gene of interest is membrane protein.

In another embodiment, the membrane (M) protein of SARS-COV-2 variant selected from alpha, beta, gamma, delta, eta, Iota, kappa, lambda and Mu.

In an embodiment, the expression construct comprises as gene of interest is envelope protein.

In preferred embodiment, the envelope (E) protein of SARS-COV-2 variant selected from alpha, beta, gamma, delta, eta, Iota, kappa, lambda and Mu.

In an embodiment, the linearization of isolated plasmid using restriction endonuclease selected from EcoRI, EcoRII, BamHI, HindIII, TaqI, NotI, HinFI, Sau3AI, PvuII*, SmaI*, HaeIII*, HgaI, AluI*, EcoRV*, EcoP15I, KpnI, PstI, SacI, SaII, ScaI*, SpeI, SphI, StuI*, BglII, XbaI, NruI.

In an embodiment, the in-vitro transcription uses cell-free system for the generation of RNA transcripts.

In vitro transcription: preparative RNA yields in analytical scale reactions (ID Pokrovskaya, V V Gurevich, Analytical Biochemistry, Volume 220, Issue 2, 1 Aug. 1994, Pages 420-423)

In an embodiment, the promoter for controlling RNA in vitro transcription based on DNA dependent RNA polymerases selected from T7, T3, and SP6 RNA polymerases.

In an embodiment, the regulatory promoter sequence is T7 polymerase.

In another embodiment, the promoter sequence selected from simian vacuolating virus 40 (SV40), cytomegalovirus (CMV), elongation factor (EF)-1, lactase LAC4, pPolh, trp, APL, AOX1, GALI, GAL10, nmt1, nmt42, nmt81 and glyceraldehyde-3-phosphate dehydrogenase (GAP).

In an embodiment, the expression vector construct comprises polyadenylation (Poly A) tail contains about 80 to about 120 adenosine nucleotides.

In an embodiment, the expression vector construct comprises polyadenylation (Poly A) tail contains 110 adenosine nucleotides.

In an embodiment, the expression vector construct comprises polyadenylation (Poly A) tail contains more than 100 adenosine nucleotides.

In an embodiment, the expression vector construct comprises SARS-COV-2 spike(S) protein, the length of spike(S) protein from about 1250 to about 1290 amino acid residues.

In an embodiment, the expression vector construct comprises SARS-COV-2 envelope (E) protein, the length of envelope (E) protein from about 50 to about 90 amino acid residues.

In an embodiment, the expression vector construct comprises SARS-COV-2 membrane (M) protein, the length of membrane (M) protein from about 200 to about 250 amino acid residues.

In an embodiment, the expression vector construct comprises SARS-COV-2 nucleocapsid (N), the length of nucleocapsid (N) protein from about 390 to about 446 amino acid residues.

In an embodiment, the purification of mRNA using chromatography method selected from high performance liquid chromatography (HPLC), low normal pressure liquid chromatography methods, gas chromatography, reversed phase HPLC (RP-HPLC), affinity chromatography, ion exchange chromatography, hydroxyapatite chromatography, core bead flow-through chromatography, Oligo dT chromatography.

In an embodiment, mRNA is purified using affinity chromatography.

In an embodiment, mRNA is purified using ion exchange chromatography.

In an embodiment, mRNA is purified using Oligo dT chromatography.

In an embodiment, mRNA is purified using high performance liquid chromatography (HPLC).

In an embodiment, mRNA is purified using mixed mode chromatography.

In an embodiment, the analysis of purified mRNA using methods selected from agarose gel electrophoresis and in-vitro cell-based expression.

### Example 1: Preparation of mRNA Vaccine Construct

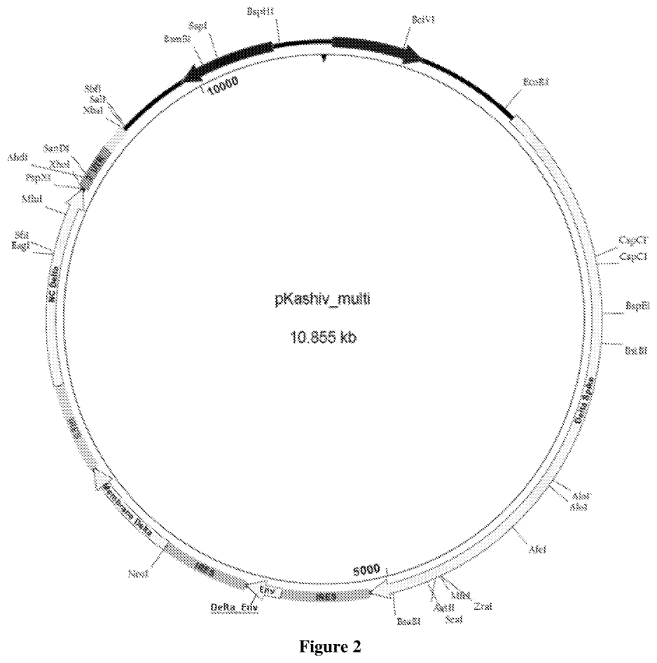

The multicistron construct is designed to have better coverage and efficiency against emerging Severe Acute Respiratory Syndrome Coronavirus 2 (SAR-COV-2). The structural sequences of virus from the database were analyzed, it is observed that other structural proteins of Severe Acute Respiratory Syndrome Coronavirus 2 as membrane, envelope and nucleocapsid apart from the spike are more conserved and less susceptible to mutation. The vaccine construct is designed to increase the breadth of immune defense against coronavirus infection as an improved mRNA vaccine. The vector construct was prepared by following the steps below:
a) The amino acid sequences of SARS-COV-2 and emerging variants were retrieved from https://covid19dashboard.regeneron.com/?tab=Home, https://marks.hms.harvard.edu/sars-cov-2/, https://www.gisaid.org/and analyzed for lineage, mutation identification for different variants.
b) Based on the amino acid sequences in the emerging mutants were reverse translated to DNA sequence which were codon optimized as per human amino acid codon usage.
c) The pKashiv_multi-DNA vector construct (10876 bp) comprises the elements as shown in FIG. 2 and respective sequence mentioned as “pKashiv_multi-DNA vector (10876 bp)” and clone into pHSG298 plasmid.
d) Prepare the clone of SARS-COV-2 delta variant sequences in the pHSG298 vector using restriction enzyme cloning.
e) Transform the E. coli host with the recombinant plasmid and further perform selection on Kanamycin containing media.
f) Screen the transform colonies for positive clone and confirm by sequencing and other identification methods known in the art.
g) Culture the recombinant E. coli cells in the desired media following by purification of plasmid DNA.
h) Linearize the plasmid DNA by restriction enzyme and then use for in vitro transcription reaction using T7 RNA polymerase.
i) Prepare the mRNA through in vitro transcription (IVT) and purify using suitable chromatography column.

### Example 2: Preparation of Plasmid DNA to Use as Template for mRNA Preparation

a) The recombinant E. coli cells were grown in the desired media in shake flask allowed to grow for 12-16 hours at 37° C. at suitable RPM, cells were harvested by centrifugation followed by cell lysis, neutralization and precipitation.
b) Purification of plasmid DNA carried out using Ion exchange chromatography.
c) Linearize the plasmid DNA by restriction enzyme and initiated in vitro transcription reaction using T7 RNA polymerase and co-transcriptional capping reagent in addition to available IVT reagents followed by DNAse treatment as shown in Table 1.
d) Purification of mRNA carried out using mixed-mode and affinity chromatography.
e) Purified mRNA was subjected to physicochemical testing such as agarose gel electrophoresis shown in FIG. 5, quantification, purity in addition to in-vitro cell-based expression as shown in FIG. 6.

TABLE 1
Reaction mixture for in vitro transcription

| | | | Final | | Amount | |
|---|---|---|---|---|---|---|
| Component | Stock Conc. | | concentration | | for 1 ml | |
| DNase/RNase-free water | | | — | | Up to 1000 μl | |
| rATP | 100 | mM | 5 | mM | 50 | μl |
| rCTP | 100 | mM | 5 | mM | 50 | μl |
| rGTP | 100 | mM | 5 | mM | 50 | μl |
| modified rUTP | 100 | mM | 5 | mM | 50 | μl |
| Co-transcriptional cap | 100 | mM | 4 | mM | 40 | μl |
| 10× transcription buffer/5× | 10×/5× | | 1× | | 100 μl/200 μl | |
| transcription buffer | | | | | | |
| DNA template linearized | (500-1000 μg/ml | | 100 | μg/ml | 100 | μg |
| | or more) | | | | | |
| Murine RNase inhibitor | 40 | U/μl | 1 | U/μl | | |
| Yeast inorganic | 0.6 | U/μl | 0.002 | U/μl | 3.33 | μl |
| pyrophosphatase | | | | | | |
| T7 RNA polymerase | 1000 | KU/ml | 8 | U/μl | 8 | μl |

| | | | |
|---|---|---|---|
| Total volume | — | 1000 | μl |

Note:
Calculate the amount of DNase/RNase-free water after obtaining the volumes of other components

1. An expression vector construct encoding more than one structural protein of severe acute respiratory syndrome coronavirus 2 (SARS-COV-2) comprising:
a) promoter sequence;
b) untranslated regions;
c) more than one structural protein selected from spike(S), envelope (E), membrane (M), and nucleocapsid (N);
d) one or more internal Ribosome Entry Site (IRES) element; and
e) polyadenylation (poly A) tail.

2. An expression vector construct encoding more than one structural protein of severe acute respiratory syndrome coronavirus 2 (SARS-COV-2) comprising:
a) promoter sequence;
b) 5′ untranslated region (5′ UTR);
c) internal ribosomal entry sites (IRES) element;
f) one or more structural proteins of SARS-COV-2 selected from spike(S), envelope (E), membrane (M), and nucleocapsid (N);
d) internal ribosomal entry sites (IRES) element;
e) 3′ untranslated region (3′ UTR); and
f) polyadenylation (Poly A) tail.

3. The expression vector construct as in claim 1 or claim 2, wherein the SARS-COV-2 variant is selected from alpha, beta, gamma, delta, eta, Iota, kappa, lambda, and Mu.

4. The expression vector construct as in claim 1, wherein the expression vector encoding more than one structural proteins of severe acute respiratory syndrome coronavirus 2 (SARS-COV-2) comprising:
a) T7 promoter;
b) 5′ Untranslated region (5′ UTR);
c) spike protein of SARS-COV-2 delta variant;
d) internal Ribosome Entry Site (IRES) element;
e) envelope protein of SARS-COV-2 delta variant;
f) internal Ribosome Entry Site (IRES) element;
g) membrane protein of SARS-COV-2 delta variant;
h) internal Ribosome Entry Site (IRES) element;
i) nucleocapsid protein of SARS-COV-2 delta variant;
j) 3′ Untranslated region (3′ UTR); and
k) poly(A) tail.

5. The expression vector construct as in claim 4 has nucleotide sequence as set forth in SEQ ID NO.: 1.

6. The expression vector construct comprises polyadenylation (Poly A) tail as in claim 1 or claim 2 or claim 4, wherein the Poly A tail contains about 80 to about 120 adenosine nucleotides.

7. The expression vector construct comprises polyadenylation (Poly A) tail as in claim 6, wherein the Poly A tail contains 110 adenosine nucleotides.

8. The expression vector construct comprises SARS-COV-2 spike(S) protein as in claim 1 or claim 2 or claim 4, wherein the length of spike(S) protein from about 1250 to about 1290 amino acid residues.

9. The expression vector construct comprises SARS-COV-2 envelope (E) protein as in claim 1 or claim 2 or claim 4, wherein the length of envelope (E) protein from about 50 to about 90 amino acid residues.

10. The expression vector construct comprises SARS-COV-2 membrane (M) protein as in claim 1 or claim 2 or claim 4, wherein the length of membrane (M) protein from about 200 to about 250 amino acid residues.

11. The expression vector construct comprises SARS-COV-2 nucleocapsid (N) protein as in claim 1 or claim 2 or claim 4, wherein the length of nucleocapsid (N) protein from about 390 to about 446 amino acid residues.

12. The expression vector construct as claimed in claim 1 or claim 2 or claim 4, expresses the mRNA capable to encode one or more structural protein provides immune response against SARS-COV-2 virus.

13. A process for the purification of mRNA comprises;
a) transfect the expression vector as claimed in claim 1, claim 2 or claim 4 into E. Coli host cells;
b) harvesting of cells followed by lysis, neutralization, and precipitation;
c) isolation and purification of plasmid;
d) linearization of purified plasmid;
e) performing the in-vitro transcription reaction for the preparation mRNA;
f) purification of mRNA by chromatography methods.

14. The purification of mRNA as claimed in claim 13 is purified using suitable chromatography selected from high performance liquid chromatography (HPLC), low normal pressure liquid chromatography method, gas chromatography, reversed phase HPLC (RP-HPLC), affinity chromatography, mixed-mode chromatography, ion exchange chromatography, hydroxyapatite chromatography, core bead flow-through chromatography, Oligo dT chromatography.


SEQUENCE LISTING (1 sequence)

SEQ ID NO: 1 - DNA, 10876 bp, synthetic construct
  source 1..10876: mol_type = other DNA
tcgacggatcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagctcgcacattcagcagcgtttttcagcgcgttttcgatcagcgtttcaatgttggtatcaacaccaggtttaactttgaacttatcggcactgacggttactgattttgaacttttgctttgccacggaacggtctgcgttgtcgggaagatgcgtgatctgatccttcaactcagcaaaagttcgccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattctaatacgactcactataaggggaaataagagagaaaagaagagtaagaagaaatataagaccccggcgccgccaccatgttcgtgttcctcgtgctgctgcccctggtgagcagccagtgcgtgaacctgcggacgcggacccagctgccgccagcctacaccaacagcttcacacggggcgtgtactaccccgacaaggtgttccggagcagcgtgctgcacagcacccaggacctgtttctgcccttcttcagcaacgtgacctggttccacgccatccacgtgtccggcaccaacgggaccaagcgcttcgacaaccccgtgctgcccttcaacgacggggtgtacttcgccagcaccgagaagtccaacatcatccgcggctggatcttcggcacgaccctggacagcaagacccagagcctgctgatcgtgaacaacgccaccaacgtggtcatcaaagtgtgcgagttccagttctgcaacgaccccttcctggacgtctactaccacaagaacaacaagagctggatggaaagcggcgtgtacagcagcgccaacaactgcaccttcgagtacgtgtcccagccgttcctgatggacctcgagggcaagcagggcaacttcaagaacctccgcgagttcgtgttcaagaacatcgacggctacttcaagatctacagcaagcacacgcccatcaacctcgtgcgggacctgccgcagggcttcagcgccctcgagccgctggtggacctgccgatcgggatcaacatcacccggtttcagaccctgctggccctgcaccggtcctacctgacgcctggcgacagcagctccggctggacagccggcgcagccgcctactacgtgggctacctgcagccgcgcaccttcctgctgaagtacaacgagaacggcaccatcaccgacgccgtggactgcgctctggacccgctgagcgagacaaagtgcaccctgaagtccttcaccgtcgagaagggcatctaccagaccagcaacttccgggtgcagcccaccgagagcatcgtgcggttcccgaacatcaccaatctgtgccccttcggcgaggtgttcaacgccacgcgcttcgcctccgtgtacgcctggaaccggaagcggatcagcaactgcgtggccgactactccgtgctgtacaactccgccagcttcagcaccttcaagtgctacggcgtcagccccaccaagctgaacgacctgtgcttcaccaacgtctacgccgacagcttcgtgatccggggcgacgaagtgcggcagatcgcccctggccagaccggcaagatcgccgactacaactacaagctgcccgacgacttcaccggctgcgtgatcgcctggaacagcaacaacctggactccaaagtcggcggcaactacaactaccgctaccggctgttccggaagtccaatctcaagcccttcgagcgggacatctccaccgagatctaccaggccggcagcaagccctgcaacggcgtcgagggcttcaactgctacttcccgctgcagagctacggcttccagccgaccaacggcgtgggctatcagccctaccgcgtggtggtgctgagcttcgagctgctgcacgcccctgccaccgtgtgcggccccaagaagtccaccaacctcgtcaagaacaaatgcgtgaacttcaacttcaacggcctgaccggcaccggcgtgctgaccgagtccaacaagaagttcctgccgttccagcagttcggccgggatatcgccgacacgacggacgccgtgcgcgacccgcagacgctcgagatcctggacatcacgccctgcagcttcggcggcgtgtccgtgatcacgcccgggaccaacaccagcaaccaggtggccgtcctgtaccagggcgtgaactgcaccgaggtgcccgtggccatccacgccgaccagctgacgcccacctggcgggtgtactccaccggctccaacgtgttccagaccagagccggctgcctcatcggggccgagcacgtcaacaacagctacgagtgcgacatccccatcggagccggcatctgcgccagctaccagacgcagacgaactcccggcggagggcccgcagcgtggccagccagagcattatcgcctacaccatgagcctgggcgccgagaactccgtggcctactccaacaactctatcgctatccccaccaacttcaccatcagcgtgacgaccgagattctgcccgtgtccatgaccaagaccagcgtcgactgcaccatgtacatctgcggcgacagcaccgagtgcagcaacctgctgctgcagtacggcagcttctgcacccagctgaatcgggccctgaccgggatcgccgtcgagcaggacaagaacacccaagaggtgttcgcccaagtgaagcagatctacaagaccccgccgatcaaggacttcggcgggttcaacttctcccagatcctgccagatccgagcaagccgtccaagcggagcttcatcgaggacctgctgttcaacaaagtgacgctggccgacgccggcttcatcaagcagtacggggactgcctgggcgacattgccgccagggacctgatctgcgcccagaagttcaacgggctcaccgtcctgccgccgctgctcaccgacgagatgatcgcccagtacacgagcgccctgctcgccgggaccatcacgtccggctggaccttcggagccggcgctgccctgcagatccccttcgccatgcagatggcctaccggttcaacggcatcggcgtgacccagaacgtgctctacgagaaccagaagctgatcgccaaccagttcaacagcgccatcggcaagatccaggacagcctgagcagcaccgccagcgctctgggcaagctgcagaacgtcgtcaaccagaacgctcaggccctgaacaccctggtcaagcagctgtcctccaacttcggcgccatcagcagcgtcctgaacgacatcctgagccggctcgacccgcctgaggccgaggtgcagatcgaccggctgatcaccggcaggctgcagtccctgcagacctacgtcacccagcagctgatccgggctgccgagatccgggcctccgccaacctggccgccacgaagatgagcgagtgcgtgctgggccagagcaagcgggtcgacttctgcggcaagggctaccacctgatgagcttcccgcagagcgcccctcacggcgtggtgttcctccacgtgacctacgtgcccgctcaagagaagaacttcacgacggccccagccatctgccacgacggcaaggctcacttccccagagaaggcgtgttcgtgtccaacggcacccattggttcgtgacgcagcggaacttctacgagccccagatcatcaccaccgacaacaccttcgtcagcggcaactgcgacgtcgtgatcggcatcgtcaacaacaccgtgtacgacccgctgcagcccgagctggactccttcaaagaggaactggacaagtacttcaagaaccacacgagccccgacgtggacctcggcgacatctccggcatcaacgccagcgtggtcaacatccagaaagagatcgaccgcctcaacgaggtggccaagaacctgaacgagagcctgatcgacctgcaagagctggggaagtacgagcagtacatcaagtggccgtggtacatctggctgggctttatcgccggcctgatcgccatcgtcatggtcaccatcatgctgtgctgcatgacgtcctgctgcagctgcctgaagggctgctgctcctgcggctcctgctgcaagttcgacgaggacgacagcgagcccgtgctgaagggcgtcaagctgcactacacctgatagacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcaacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatgccgccaccatgtacagcttcgtgtccgaggaaacgggcaccctgatcgtcaactccgtcctcctgttcctggccttcgtcgtgttcctgctggtcaccctggccatcctgacggccctccggctgtgcgcctactgctgcaacatcgtgaacgtgtccctcgtgaagcccagcttctacgtgtacagccgcgtgaagaacctcaacagcagccgggtgccagacctgctggtgtaatgaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcaacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatgccgccaccatggccgacagcaacgggaccatcaccgtcgaggaactgaagaaactcctcgagcagtggaacctcgtgatcggcttcctcttcctgacctggatctgcctgctccagttcgcctacgccaaccgcaaccggttcctgtatatcatcaagctgatctttctgtggctgctgtggcccgtgacgctcgcctgcttcgtgctggccgccgtgtaccggatcaactggatcaccggcgggatcgccactgccatggcctgcctcgtgggcctgatgtggctgagctacttcattgccagcttccgcctcttcgcccggacgcggtccatgtggtccttcaatcccgagacaaacatcctgctcaacgtgcccctgcacggcacgatcctgactcggcccctgctcgagagcgagctggtcatcggcgccgtgatcctgcggggccacctccggatcgccgggcaccacctgggccgctgcgacatcaaggatctgcccaaagagatcacggtcgccacctctcggaccctgagctactacaagctgggcgccagccagcgcgtggccggcgacagcggcttcgccgcctacagccgctaccgcatcgggaactacaagctgaacaccgaccacagctccagcagcgacaatatcgccctcctggtgcagtgatgaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcaacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatgccgccaccatgagcgacaacgggccccagaaccagcgcaacgcccctcggatcaccttcggcggaccctccgacagcaccggcagcaaccagaacggcgagcggagcggcgccaggtccaagcagcgcaggccccaggggctgccgaacaacaccgccagctggttcaccgctctcacccagcacggcaaagagggcctgaagttcccgcgaggccagggcgtccccatcaacaccaacagcagccccgacgaccagatcggctactacaggcgggccacgcggcggatcagaggcggcgacgggaagatgaaggatctgagcccgcggtggtacttctactacctcggcactggccccgaggccgggctgccctacggcgccaacaaggacggcatcatctgggtcgccaccgagggcgccctgaacacgcccaaggaccacatcggcacgcgcaacccggccaacaacgccgccatcgtcctgcagctgccccagggcaccacgctgcccaagggcttctacgccgagggctcccgcggaggcagccaggccagcagccgcagctcctctcggtcccgcaacagctcccggaacagcacgcccggcagcagcatgggcacgagcccggcacggatggccggcaacggctgcgacgccgctctggccctcctcctgctggaccgcctgaaccagctcgagtccaagatgtccggcaagggccagcagcagcaggggcagaccgtgaccaagaagagtgccgccgaggcctccaagaagccccggcagaagcggaccgccaccaaggcctacaacgtgacccaggccttcggccgcaggggccctgagcagacccaggggaacttcggggaccaagagctgatccggcagggcaccgactacaagcactggccgcagatcgcccagttcgccccgagcgccagcgccttcttcggcatgagccggatcggcatggaagtgacgcccagcggcacctggctgacctacaccggggccatcaagctcgacgacaaggacccgaacttcaaggaccaagtgatcctcctgaacaagcacatcgacgcctacaagacgttcccgccgaccgagccgaagaaggacaagaagaagaaggcctacgagacacaggccctgccgcagcgccagaagaagcagcagacggtcacgctgctgccagccgccgacctggacgacttcagcaagcagctccagcagagcatgagcagcgccgacagcacgcagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaaagcataaccccttggggcctctaaacgggtcttgaggggttttttgtcgcgatctagatcgcgatctagaggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctc